(19) United States
(12) Patent Application Publication
(10) Pub. No.: US 2006/0079421 A1
(43) Pub. Date: Apr. 13, 2006

(54) Stable multi-phased personal care composition

(76) Inventors: Julie Ann Wagner, Cincinnati, OH (US); Karl Shiqing Wei, Mason, OH (US); Edward Dewey Smith III, Mason, OH (US); Sanjeev Midha Midha, Mason, OH (US); James Merle Heinrich, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/198,618

(22) Filed: Aug. 5, 2005

Related U.S. Application Data
(60) Provisional application No. 60/617,392, filed on Oct. 8, 2004.
(60) Provisional application No. 60/628,076, filed on Nov. 15, 2004.

(51) Int. Cl. A61K 8/00

(52) U.S. Cl. 510/130

(57) ABSTRACT

The present invention is a stable multi-phase personal care composition comprising: at least two visually distinct phases; wherein at least one visually distinct phase comprises a cleansing phase comprising from about 2% to about 90%, by weight of the cleansing phase, of a surfactant component; and wherein said cleansing phase comprises a polymeric phase structurant; and wherein said visually distinct phases form a pattern.

### CROSS-REFERENCE TO RELATED APPLICATIONS

This application claims the benefit of Provisional Application Ser. No. 60/617,392 (Case 9791P), filed Oct. 8, 2004, and Provisional Application Ser. No. 60/628,076 (Case 9833P), filed Nov. 15, 2004.

### FIELD OF THE INVENTION

The present invention relates a stable multi-phase personal care composition comprising: at least two visually distinct phases; wherein at least one visually distinct phase comprises a cleansing phase comprising from about 2% to about 90%, by weight of the cleansing phase, of a surfactant component; and wherein said cleansing phase comprises a polymeric phase structurant; and wherein said visually distinct phases form a pattern.

### BACKGROUND OF THE INVENTION

Personal care compositions are becoming more popular in the United States and around the world. Personal care compositions are well known and widely used. Desirable personal care composition must meet a number of criteria. For example, in order to be acceptable to consumers, a personal care composition must exhibit good cleaning properties, must exhibit good lathering characteristics, must be mild to the skin (not cause drying or irritation) and preferably should even provide a conditioning benefit to the skin. Personal care compositions have also been used to alter the color and appearance of skin.

Personal care compositions that attempt to provide skin-conditioning benefits are known. Many of these compositions are aqueous systems comprising an emulsified conditioning oil or other similar materials in combination with a lathering surfactant. Although these products provide both conditioning and cleansing benefits, it is often difficult to formulate a product that deposits sufficient amount of skin conditioning agents on skin during use. In order to combat emulsification of the skin conditioning agents by the cleansing surfactant, large amounts of the skin conditioning agent are added to the compositions. However, this introduces another problem associated with these dual cleansing and conditioning products. Raising the level of skin conditioning agent in order to achieve increased deposition negatively affects product lather performance and stability. To combat the reduction in lather performance, often the level of surfactant is increased, negatively affecting skin conditioning. While it is generally desirable to reduce surfactant levels to improve mildness, it is often not possible to do so and maintain stability and lather performance, especially in the presence of a hydrophobic benefit component.

One attempt at providing conditioning and cleansing benefits from a personal cleansing product while maintaining stability has been the use of dual-chamber packaging. These packages comprise separate cleansing compositions and conditioning compositions, and allow for the co-dispensing of the two in a single or dual stream. The separate conditioning and cleansing compositions thus remain physically separate and stable during prolonged storage and just prior to application, but then mix during or after dispensing to provide conditioning and cleansing benefits from a physically stable system. Although such dual-chamber delivery systems provide improved conditioning benefits over the use of conventional systems, it is often difficult to achieve consistent and uniform performance because of the uneven dispensing ratio between the cleansing phase and the conditioning phase from these dual-chamber packages. Additionally, these packaging systems add considerable cost to the finished product.

Accordingly, the need still remains for stable multi-phased personal care composition that provides cleansing with increased lather longevity and improved lathering characteristics, and skin benefits such as silky skin feel, improved soft skin feel, and improved smooth skin feel. The need also remains for a personal care composition comprising two phases in physical contact that remain stable for long periods of time.

It is therefore an object of the present invention to provide a stable multi-phased personal care composition comprising visually distinct phase comprising a surfactant having a structured domain combined with a second visually distinct phase that can comprise high levels of benefit components that are not emulsified in the composition but comprised in a separate benefit phase so that the benefit components can be deposited at higher levels while at the same time maintaining superior lather performance, stability and mildness.

### SUMMARY OF THE INVENTION

The present invention relates a stable multi-phase personal care composition comprising: at least two visually distinct phases; wherein at least one visually distinct phase comprises a cleansing phase comprising from about 2% to about 90%, by weight of the cleansing phase, of a surfactant component; and wherein said cleansing phase comprises a polymeric phase structurant; and wherein said visually distinct phases form a pattern.

The present invention further relates to a stable multi-phase personal care composition comprising: at least two visually distinct phases; wherein at least one visually distinct phase comprises a cleansing phase comprising from about 2% to about 90%, by weight of the cleansing phase, of a surfactant component; and wherein said cleansing phase has a Structured Domain Volume Ratio of at least about 45%.

The present invention further relates to a multi-phase personal care composition comprising: at least two visually distinct phases comprising; a first phase comprising a cleansing phase comprising from about 2% to about 90%, by weight of said cleansing phase, of a surfactant selected from the group consisting of anionic surfactant, nonionic surfactant, zwitterionic surfactant, cationic surfactant, soap, and mixtures thereof; wherein said cleansing phase is non-Newtonian shear thinning, has a viscosity of equal to or greater than about 3,000 cps; a benefit phase comprising a hydrophobic composition comprising from about 1% to about 100%, by weight of said benefit phase of a hydrophobic material is selected from the group consisting of lipids, hydrocarbons, fats, oils, hydrophobic plant extracts, fatty acids, essential oils, silicone oils, and mixtures thereof; wherein a weight ratio between said cleansing phase and said benefit phase is from about 1:99 to about 99:1 and said cleansing phase and benefit phase are in physical contact in the same package and remain stable in ambient conditions for at least about 180 days; and wherein said cleansing phase and benefit phase form a pattern; and wherein at least one phase comprises a polymeric phase structurant; and wherein said visually distinct phase are stable.

The present invention further relates to a multi-phase personal care composition comprising: at least two visually distinct phases comprising; a first phase comprising a cleansing phase comprising from about 2% to about 90%, by weight of the cleansing phase, of a surfactant selected from the group consisting of anionic surfactant, non-ionic surfactant, zwitterionic surfactant, cationic surfactant, soap and mixtures thereof; wherein said cleansing phase is non-Newtonian shear thinning, has a viscosity of equal to or greater than about 3,000 cps; and a separate non-lathering structured aqueous phase; and wherein the ratio of the cleansing phase to the non-lathering structured aqueous phase is from about 90:1 to about 1:90; wherein the cleansing phase and non-lathering structured aqueous phase are present as a pattern; and wherein at least one phase comprises a polymeric phase structurant; and wherein said visually distinct phase are stable.

The present invention is also directed to a method of cleansing, moisturizing and delivering skin benefit agents and particles to the skin by applying to the skin a composition as described above.

### DETAILED DESCRIPTION OF THE INVENTION

The present invention relates a stable multi-phase personal care composition comprising: at least two visually distinct phases; wherein at least one visually distinct phase comprises a cleansing phase comprising from about 2% to about 90%, by weight of the cleansing phase, of a surfactant component; and wherein said cleansing phase comprises a polymeric phase structurant; and wherein said visually distinct phases form a pattern. These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water.

The term “ambient conditions” as used herein, refers to surrounding conditions at one (1) atmosphere of pressure, 50% relative humidity, and 25° C.

The term “cosmetically efficacious level” as used herein, is a level conferring a benefit during use of the composition.

The term “Consistency value” or “k” as used herein is a measure of viscosity and is used in combination with Shear Index, to define viscosity for materials whose viscosity is a function of shear. The measurements are made at 25° C. and the units are poise (equal to 100 centipoise).

The term “domain”, as used herein means a volume of material, component, composition or phase comprising a molecular mixture which can be concentrated but not further separated by physical forces such as ultracentrifugation. For example, surfactant lamellar, surfactant micellar, surfactant crystal, oil, wax, water-glycerine mixture, hydrated hydrophilic polymer all constitute domains which can be concentrated and observed by ultracentrifugation, but which cannot be further separated into distinct molecular components by the same forces.

The term “hydrophobically modified interference pigment” or “HMIP”, as used herein, means a portion of the interference pigment surface has been coated, including both physical and chemical bonding of molecules, with a hydrophobic material.

The term “interference pigment”, as used herein, means a pigment with pearl gloss prepared by coating the surface of a particle substrate material (generally platelet in shape) with a thin film. The thin film is a transparent or semitransparent material having a high refractive index. The higher refractive index material shows a pearl gloss resulting from mutual interfering action between reflection and incident light from the platelet substrate/coating layer interface and reflection of incident light from the surface of the coating layer.

By the term “liquid crystalline phase inducing structurant” as used herein, is meant a component which is compatible with the surfactant components and induces the formation of and/or increases the percentage of a lamellar phase, preferably by directly participating in lateral arrangement of the surfactant molecules. By the term “multi-phased” or “multi-phase” as used herein, is meant that the at least two phases herein occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phased” personal care compositions comprising at least two visually phases are present within the container as a visually distinct pattern. The pattern results from the mixing or homogenization of the “multi-phased” composition. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled and combinations thereof.

In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The size of the stripes can be at least about 0.1 mm in width and 10 mm in length, preferably at least about 1 mm in width and at least 20 mm in length. The phases may be various different colors and/or include particles, glitter or pearlescent agents in at least one of the phases in order to offset its appearance from the other phase(s) present.

The term “stable multi-phased personal care composition” as used herein, refers to compositions intended for topical application to the skin or hair which essentially retains their appearance and character during use, i.e., shaking is not required nor is complex packaging or dispensing means.

The term “phases” as used herein, refers to a region of a composition having one average composition, as distinct from another region having a different average composition, wherein the regions are visible to the naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase may also be constituted or re-constituted into a bulk phase in order to observe its properties, e.g., by centrifugation, filtration or the like.

The term “stable” as used herein, unless otherwise specified, refers to compositions that maintain at least two “separate” phases when sitting in physical contact at ambient conditions for a period of at least about 180 days wherein the distribution of the two phases in different locations in the package does not change over time. By “separate” is meant that the well-distributed nature of the visually distinct phases and also the pattered appearance is compromised, such that larger regions of at least one phase collect until the balanced dispensed ratio of the two or more compositions relative to each other is compromised.

The term “opaque” structured domain as used herein refers to a surfactant containing domain with ordered structures (e.g., lamellar structure, vesicule structure, cubic structure, etc.) and it is visually opaque to a naked eye in a 10 mm inner diameter plastic centrifuge tube after the Ultracentrifugation Method described herein. Opaque structured domains in the cleansing phase include opaque surfactant-polymer domains having structure.

The term “Shear Index” or “n” as used herein is a measure of viscosity and is used in combination with Consistency value, to define viscosity for materials whose viscosity is a function of shear. The measurements are made at 25° C. and the units are dimensionless.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The Vaughan Solubility Parameter (VSP) as used herein is a parameter used to define the solubility of hydrophobic compositions comprising hydrophobic materials. Vaughan Solubility parameters are well known in the various chemical and formulation arts and typically have a range of from about 5 to about 25 (cal/cm³)^{1/2}.

The term “Zero Shear Viscosity” as used herein is a measure of viscosity of a cleansing phase in the low stress region, prior to the onset of flow, as determined by the method herein.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The mild multi-phased personal care composition compositions and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

### Product Form

The stable multi-phased personal care composition of the present invention is typically in the form of a liquid. The term “liquid” as used herein means that the composition is generally flowable to some degree. “Liquids”, therefore, can include liquid, semi-liquid, cream, lotion or gel compositions intended for topical application to skin. The compositions typically exhibit a viscosity of from about 1,500 cps to about 1,000,000 cps, as measured by the Viscosity Method as described in copending application Ser. No. 60/542,710 filed on Feb. 6, 2004. These compositions contain at least two phases, which are described in greater detail hereinafter.

When evaluating a stable multi-phased personal care composition, by the methods described herein, preferably each individual phase is evaluated prior to combining, unless otherwise indicated in the individual methodology. However, if the phases are combined, each phase can be separated by centrifugation, ultracentrifugation, pipetting, filtering, washing dilution, concentration, or combination thereof, and then the separate components or phases can be evaluated. Preferably, the separation means is chosen so that the resulting separated components being evaluated is not destroyed, but is representative of the component as it exists in the stable multi-phased personal care composition, i.e., its composition and distribution of components therein is not substantially altered by the separation means. Generally, patterned multi-phase compositions comprise domains significantly larger than colloidal dimensions so that separation of the phases into the bulk is relatively easy to accomplish while retaining the colloidal or microscopic distribution of components therein. All of the product forms contemplated for purposes of defining the compositions and methods of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

In a preferred embodiment of the present invention the stable multi-phased personal care composition, the composition has at least two visually distinct phases wherein at least one phase is visually distinct from a second phase. The visually distinct phases are packaged in physical contact with one another and are stable.

### Phases

The stable multi-phase personal care compositions of the present invention comprise at least two visually distinct phases, wherein the composition can have a first phase a second phase and so on. The ratio of a first phase to a second phase is about 1:99 to about 99:1, preferably 90:10 to about 10:90, more preferably about 80:20 to about 20:80, even more preferably about 70:30 to about 30:70, still even more preferably about 60:40 to about 40:60, even still even more preferably about 50:50. Each phase could be one or more of the following nonlimiting examples including: a cleansing phase, a benefit phase, and a non-lathering structured aqueous phase, which are described in greater detail hereinafter. When the cleansing phase is present with a second phase the ratio of the cleansing phase to the second phase is from about 99:1 to about 1:99. The ratio of the cleansing phase to the second phase is preferably from about 50:50, more preferably from about 70:30 to about 30:70, even more preferably from about 80:20 to about 20:80, still even more preferably from about 90:10 to about 10:90.

Cleansing Phase

The stable multi-phase personal care composition of the present invention can comprise a cleansing phase. The cleansing phase comprises a surfactant component or mixtures of surfactants. The stable multi-phased personal care composition comprises from about 1% to about 99%, by weight of the composition, of said cleansing phase.

The cleansing phase comprising the surfactant component preferably has structure. Yield Stress and Zero Shear Viscosity are useful properties for cleansing phases having structure which yield upon application of stress, such as the stress to dispense the composition from a package. The Yield Point is the amount of stress required to initiate flow of the cleansing phase, and the Zero Shear Viscosity is the median viscosity in the stress region prior to the onset of flow, as determined by the methods described herein. The cleansing phase provides a Yield Point of greater than about 0.5 Pascal, preferably greater than about 1.0 Pascal, more preferably greater than 1.5 Pascal, even more preferably greater than about 2 Pascal. The cleansing phase provides a Zero Shear Viscosity at least about 500 Pa-s, preferably at least about 1,000 Pa-s, more preferably at least about 1,500 Pa-s, even more preferably at least about 2,000 Pa-s.

Surfactant Component

The surfactant component comprises a surfactant or a mixture of surfactants. The surfactant component comprises surfactants suitable for application to the skin or hair. Suitable surfactants for use herein include any known or otherwise effective cleansing surfactant suitable for application to the skin, and which is otherwise compatible with the other essential ingredients in the stable multi-phased personal care composition including water. These surfactants include anionic, nonionic, cationic, zwitterionic or amphoteric surfactants, soap or combinations thereof.

The stable multi-phased personal care composition preferably comprises a surfactant component at concentrations ranging from about 2% to about 90%, more preferably from about 5% to about 40%, even more preferably from about 10% to about 30%, still more preferably from about 12% to about 25%, still even more preferably from about 13% to about 25%, and even still even more preferably from about 14% to about 18.5%, by weight of the cleansing phase. The preferred pH range of the stable multi-phased personal care composition is from about 5 to about 8. The surfactant component in the present invention exhibits Non-Newtonian shear thinning behavior.

The cleansing phase comprising the surfactant component comprises a structured domain comprising a structured surfactant system. The structured domain enables the incorporation of high levels of benefit components in a separate phase that are not emulsified in the composition. In a preferred embodiment the structured domain is an opaque structured domain. The opaque structured domain is preferably comprises a lamellar phase. The lamellar phase produces a lamellar gel network. The lamellar phase provides resistance to shear, adequate yield to suspend particles and droplets and at the same time provides long term stability, since it is thermodynamically stable. The lamellar phase has a higher viscosity without the need for viscosity modifiers. The cleansing phase comprising the surfactant component has a Structured Domain Volume Ratio of at least about 45%, preferably at least about 50%, more preferably at least about 55%, even more preferably at least about 60%, still more preferably at least about 65%, still even more preferably at least about 70%, and still even still more preferably at least about 80% as measured by the Ultracentrifugation Method described hereafter.

The cleansing phase has a Total Lather Volume of at least about 600 ml, preferably greater than about 800 ml, more preferably greater than about 1000 ml, even more preferably greater than about 1200 ml, and still more preferably greater than about 1500 ml, as measured by the Lather Volume Test described hereafter. The surfactant component preferably has a Flash Lather Volume of at least about 300 ml, preferably greater than about 400 ml, even more preferably greater than about 500 ml, as measured by the Lather Volume Test described hereafter.

The structured domain has a Total Lather Volume of at least about 450 ml, preferably greater than about 500 ml, more preferably greater than about 600 ml, even more preferably greater than about 800 ml, still more preferably greater than about 1000 ml, and still even more preferably greater than about 1250 ml, as measured by the Lather Volume Test described hereafter. The structured domain preferably has a Flash Lather Volume of at least about 200 ml, preferably greater than about 250 ml, even more preferably greater than about 300 ml, as measured by the Lather Volume Test described hereafter.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678.

Anionic surfactants suitable for use in the cleansing phase include alkyl and alkyl ether sulfates. These materials have the respective formula ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohols having from about 8 to about 24 carbon atoms. Preferably, R has from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with about 1 to about 10, preferably from about 3 to about 5, and more preferably with about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, preferably about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Preferred anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, sodium cocoyl isethionate, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants may be used in some embodiments.

Additional surfactant from the classes of amphoteric, zwitterionic surfactant, cationic surfactant, and/or nonionic surfactant may be incorporated in the cleansing phase compositions.

Amphoteric surfactants suitable for use in the cleansing phase include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Zwitterionic surfactants suitable for use in the cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula: 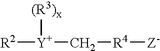
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine; lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Amphoacetates and diamphoacetates may also be used. 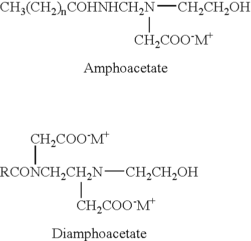
Amphoacetates and diamphoacetates conform to the formulas (above) where R is an aliphatic group of 8 to 18 carbon atoms. M is a cation such as sodium, potassium, ammonium, or substituted ammonium. Sodium lauroamphoacetate, sodium cocoamphoactetate, disodium lauroamphoacetate, and disodium cocodiamphoacetate are preferred in some embodiments.

Cationic surfactants can also be used in the cleansing phase, but are generally less preferred, and preferably represent less than about 5% by weight of the compositions.

Suitable nonionic surfactants for use in the aqueous cleansing phase include condensation products of alkylene oxide groups (hydrophilic in nature) with an organic hydrophobic compound, which may be aliphatic or alkyl aromatic in nature.

In an alternate embodiment of the present invention the cleansing phase comprises a surfactant component comprising a mixture of at least one nonionic surfactant, at least one anionic surfactant and at least one amphoteric surfactant, and an electrolyte.

Non-Ionic Surfactants

In an alternate embodiment of the present invention the stable multi-phased personal care composition can comprises at least one nonionic surfactant. Preferably the nonionic surfactant has an HLB from about 1.0 to about 15.0, preferably from about 3.4 to about 15.0, more preferably from about 3.4 to about 9.5, even more preferably from about 3.4 to about 5.0. The multi-phased personal care composition preferably comprises a nonionic surfactant at concentrations ranging from about 0.01% to about 50%, more preferably from about 0.10% to about 10%, and even more preferably from about 0.5% to about 5.0%, by weight of the surfactant component.

Nonionic surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, and mixtures thereof. In a preferred embodiment the nonionic surfactant is selected from the group consisting of glyceryl monohydroxystearate, Steareth-2, hydroxy stearic acid, propylene glycol stearate, PEG-2 stearate, sorbitan monostearate, glyceryl stearate, glyceryl laurate, laureth-2 and mixtures thereof. In a preferred embodiment the nonionic surfactant is Steareth-2.

Nonionic surfactants also useful herein include, lauramine oxide, cocoamine oxide.

Anionic Surfactants

In the alternate embodiment of the present invention the stable multi-phased personal care composition can comprises at least one anionic surfactant. Nonlimiting examples of suitable anionic surfactant were discussed previously.

Amphoteric Surfactants

In the alternate embodiment of the present invention the stable multi-phased personal care composition can comprises at least one amphoteric surfactant. Nonlimiting examples of suitable amphoteric surfactant were discussed previously.

Electrolyte

The electrolyte, if used, can be added per se to the stable multi-phased personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride or sodium or ammonium sulfate. A preferred electrolyte is sodium chloride. The electrolyte is preferably added to the surfactant component of the composition.

The electrolyte, when present, should be present in an amount, which facilitates formation of the stable composition (Non-Newtonian shear thinning behavior). Generally, this amount is from about 0.1% by weight to about 15% by weight, preferably from about 1% to about 6% by weight of the multi-phased personal care, but may be varied if required.

In another alternative embodiment of the present invention, the surfactant for use in the cleansing phase can be mixtures of surfactants. Suitable surfactant mixtures can comprise water, at least one anionic surfactant as described previously, an electrolyte as described previously, and at least one alkanolamide. The alkanolamide if present has the general structure of: 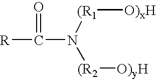
wherein R is C₈ to C₂₄, or preferably in some embodiments C₈ to C₂₂ or in other embodiments C₈ to C₁₈, saturated or unsaturated, straight chain or branched, aliphatic group; R₁ and R₂ are the same or different C₂-C₄ straight chain or branched aliphatic group; x is from 0 to 10; y is from 1 to 10; and wherein the sum of x and y is less than or equal to 10.

The amount of alkanolamide in the composition is typically about 0.1% to about 10%, by weight of the lathering cleansing phase, and in some embodiments is preferably from about 2% to about 5%, by weight of the lathering cleansing phase. Suitable alkanolamides include Cocamide MEA (Coco monethanolamide) and Cocamide MIPA (Coco monoisopropranolamide).

Polymeric Phase Structurant

The cleansing phase of the stable multi-phase personal care composition can comprise a polymeric phase structurant. The compositions of the present invention comprise from about 0.05% to about 10%, preferably from about 0.1% to about 4% and more preferably from about 0.2% to about 2% of a polymeric phase structurant. Nonlimiting examples of polymeric phase structurant include but is not limited to the following examples: deflocculating polymers, naturally derived polymers, synthetic polymers, crosslinked polymers, block polymers, block copolymers, copolymers, hydrophilic polymers, nonionic polymers, anionic polymers, hydrophobic polymers, hydrophobically modified polymers, associative polymers, oligomers, and copolymers thereof.

The polymeric phase structurant may also beneficially act in conjunction with other components of the cleansing phase or benefit phase or non-lathering structured aqueous phase an inclusive or exclusive manner, for example to form a distinct polymer rich sub-phase in the cleansing phase to enhance stability of the composition, improve mildness of the composition, increase deposition from the composition onto the skin. Such phases can broadly be considered coacervates and/or flocs, especially if they form upon dilution of the composition or the cleansing phase, and are observable by simple dilution and observation, such as a 5-10% dilution of the cleansing phase in water which can be centrifuged lightly. While theory provides considerable support for specific coacervates and technologies comprising them have been commercialized, such as cationic polymer-anionic surfactant interactions, polymer interactions are complex and governed by enthalpic and entropic interactions between molecules, macromolecules, electrolytes, ions and solvent molecules which do not preclude the formation of flocs, phases, coacervates and the like from a variety of components leading to the aforementioned benefits in cleansing phases and compositions disclosed herein, either at their full strength (e.g., in the package) or upon dilution (e.g., during use). The existence of said flocs, coacervates, or phases is not intended to be limited by theory or existing nomenclature.

Preferably the polymeric phase structurant comprises a first monomer and a second monomer, wherein the first monomer is selected from the group consisting of acrylic acid, salts of acrylic acid, C1-C4 alkyl-substituted acrylic acid, salts of C1-C4 alkyl-substituted acrylic acid, C1-C4 alkyl esters of acrylic acid, C1-C4 alkyl esters of C1-C4 alkyl-substituted acrylic acid, maleic anhydride, and mixtures thereof; and the monomer is a long chain ester monomer selected from the group consisting of C10-C30 alkyl esters of acrylic acid, C10-C30 alkyl esters of C1-C4 alkyl-substituted acrylic acid, and mixtures thereof. The salts of the acids described in the previous sentence are selected from the group consisting of alkali metal salts, alkaline metal salts, ammonium salts, and mono-, di-, tri-, and tetra-alkyl ammonium salts. The C1-C4 alkyl-substituted acrylic acids described in the first sentence of this paragraph include methacrylic acids, ethacrylic acids, and the like, wherein the alkyl substituent can be either on the C2 or C3 position of the acid molecule. The C₁-C₄ alkyl esters described in the first sentence in this paragraph include methyl and ethyl esters as well as branched C3 and C4 esters.

Preferably these polymeric phase structurant are crosslinked and further comprise a crosslinking agent that is a polyalkenyl polyether of a polyhydric alcohol containing more than one alkenyl ether group per molecule, wherein the parent polyhydric alcohol contains at least 3 carbon atoms and at least 3 hydroxyl groups. Preferred crosslinking agents are those selected from the group consisting of allyl ethers of sucrose and allyl ethers of pentaerythritol, and mixtures thereof. These polymeric phase structurant useful in the present invention are more fully described in U.S. Pat. No. 5,087,445, to Haffey et al., issued Feb. 11, 1992; U.S. Pat. No. 4,509,949, to Huang et al., issued Apr. 5, 1985, U.S. Pat. No. 2,798,053, to Brown, issued Jul. 2, 1957; which are incorporated by reference herein in their entirety. See also, CTFA International Cosmetic Ingredient Dictionary, fourth edition, 1991, pp. 12 and 80; which is also incorporated herein by reference in its entirety.

Specific examples of naturally derived polymers which can be used in the cleansing phase are starch and starch derivates such as amylose and amylopectin, starch hydroxypropylphosphate, strach octenyl succinate; marine gums such as alginates and algin derivatives such as propylene glycol alginate; pectins such as high methoxy pectin; food and plant gums such as carageenans, gum arabic or acacia gums, guar gum, locust bean gum; biosaccharides such as xanthan gum; shellfish saccharides such as chitosan and its derivates; cellulose derivatives such as methlcellulose, ethylcellulose, hydroxypropylcellulose, hydroxyethylcellulose and other cellulose derivatives; gelatin, casein and other proteins.

Examples of commercially available polymeric phase structurant, synthetic polymers and copolymers which can be used in the cleansing phase are water soluble synthetic polymers such as polyacrylates, polymethacrylates, polyacrylamides, polymethacrylamides, polyurethanes, polyesters, polyethers, polyvinylalcohols, polyalkylene oxide alkyl ethers such as PPG-15 decyl ether, vinyl esters such as polyvinylpyrrolidone, Pemulen TR-1, Pemulen TR-2, ETD 2020, Carbopol 1382 (Acrylates/C10-30 alkyl acrylate crosspolymer-Noveon), Carbopol 940, Carbopol 980, Carbopol 954, Carbopol Aqua SF-1, Carbomer, Acrylates/Acrylamide Copolymers, Acrylates Copolymers, Acrylates Crosspolymers, Acrylates/Acrylamide Copolymers, Acrylates/Acrylamide Crosspolymers, Acrylates/Alkyl Acrylates Copolymers, Acrylates/Alkyl Acrylates Crosspolymers, Acrylates/VA Copolymers, Acrylates/VA Crosspolymers, Aminoalkyl and aminoalkanol Acrylates Copolymers, Acrylates/Dimethicone Copolymers, Acrylates/Dimethicone Crosspolymers, Acrylamide/Acrylate Copolymers, Acrylamide/Acrylate Crosspolymers, Ammonium Polyacrylate, Ammonium Acrylates Copolymer, Sodium Polyacrylate Starch, TEA-Acrylates Copolymers, TEA-Acrylates Crosspolymers, Natrosol CS Plus 330, 430, Polysurf 67 (cetyl hydroxyethyl cellulose-Hercules), Aculyn 22 (acrylates/steareth-20 methacrylate copolymer—Rohm & Haas) Aculyn 25 (acrylates/laureth-25 methacrylate copolymer—Rohm & Haas), Aculyn 28 (acrylates/beheneth-25 methacrylate copolymer—Rohm & Haas), Aculyn 46 (PRG-150/stearyl alcohol/SMDI copolymer—Rohm & Haas) Stabylen 30 (acrylates/vinyl isodecanoate-3V), Structure 2001 (acrylates/steareth-20 itaconate copolymer-National Starch), Structure 3001 (acrylates/ceteth-20 itaconate copolymer-National Starch), Structure Plus (acrylates/aminoacrylates/C10-30 alkyl PEG 20 itaconate copolymer-National Starch), Structure XL, Quatrisoft LM-200 (polyquaternium-24) and mixtures and copolymers of hydrophilic polymers with either hydrophilic or hydrophobic side chains.

Specific examples of hydrophilic polymers which can be used in the cleansing phase are starches, celluloses, polyacrylates, polyacrylamides, xanthan gum and copolymers and derivatives thereof.

Liquid Crystalline Phase Inducing Structurant

The cleansing phase of the present compositions optionally, but preferably, further comprise a liquid crystalline phase inducing structurant when present is at concentrations ranging from about 0.3% to about 15% by weight of the cleansing phase, more preferably at from about 0.5% to about 5% by weight of the cleansing phase. Not being bound by theory, the liquid crystalline phase inducing structurant functions in the compositions to form a thermodynamic domain, preferably a lamellar (structured) domain. It is believed the lamellar domain enhances the interfacial stability between the phases of the present compositions.

Suitable liquid crystalline phase inducing structurant include a fatty acid or ester derivatives thereof, a fatty alcohol, trihydroxystearin (available from Rheox, Inc. under the trade name THIXCIN® R). Additional nonlimiting examples of fatty acids which may be used are C₁₀-C₂₂ acids such as the following: lauric acid, oleic acid, isostearic acid, linoleic acid, linolenic acid, ricinoleic acid, elaidic acid, arichidonic acid, myristoleic acid and palmitoleic acid, and the like. Ester derivatives include propylene glycol isostearate, propylene glycol oleate, glyceryl isostearate, glyceryl oleate and polyglyceryl diisostearate, propylene, glycol dilaurate, and the like. Preferably, the liquid crystalline phase inducing structurant is selected from lauric acid or trihydroxystearin.

Benefit Phase

The stable multi-phase personal care compositions of the present invention can comprise a benefit phase. The benefit phase in the present invention is preferably anhydrous. The benefit phase comprises hydrophobic compositions comprising hydrophobic materials. The benefit phase comprises from about 1% to about 100%, preferably at least about 35%, most preferably at least about 50% of a hydrophobic material. The hydrophobic compositions suitable for use in the present invention have a Vaughan Solubility Parameter of from about 5 to about 15. The hydrophobic compositions are preferably selected among those having defined rheological properties as described hereinafter, including selected Consistency value (k) and Shear Index (n). These preferred rheological properties are especially useful in providing the stable multi-phased personal care compositions with improved deposition of hydrophobic materials on the skin.

Vaughan Solubility Parameter Value (VSP)

The hydrophobic compositions for use in the benefit phase of the stable multi-phase personal care composition has a Vaughan Solubility Parameter (VSP) of from about 5 to about 15, preferably from about 5 to about 10, more preferably from about 6 to about 9. These solubility parameters are well known in the formulation arts, and are defined by Vaughan in Cosmetics and Toiletries Vol. 103, p 47-69, October 1988.

Non-limiting examples of hydrophobic materials having VSP values ranging from about 5 to about 15 include the following:

Vaughan Solubility Parameters*

| | |
|---|---|
| Cyclomethicone | 5.92 |
| Squalene | 6.03 |
| Petrolatum | 7.33 |
| Isopropyl Palmitate | 7.78 |
| Isopropyl Myristate | 8.02 |
| Castor Oil | 8.90 |
| Cholesterol | 9.55 |

As reported in Solubility, Effects in Product, Package, Penetration and Preservation, C. D. Vaughan, Cosmetics and Toiletries, Vol. 103, October 1988.

Rheology

Skin Feel Rheology is used to determine the preferred rheology profile of the benefit phase so that when the stable multi-phased personal care composition is deposited on the skin, the skin feels moisturized but not heavy or sticky or draggy. The consistency value is a measure of the skin feel of the benefit phase as defined by Consistency Value (K) and Shear Index (n). The benefit phase has a Consistency Value (K) from about 30 to about 350 Pa-s, preferably from about 35 to about 300 Pa-s, more preferably from about 40 to about 250 Pa-s, still more preferably from about 45 to about 150 Pa-s and even still more preferably from about 15 to about 125 Pa-s. The benefit phase has a Shear Index from about 0.025 to about 0.93, preferably from about 0.05 to about 0.70 and more preferably from about 0.09 to about 0.60. The values are determined at 25° C.

The benefit phase can be characterized by Consistency Value (K) and Shear Index (n) values as defined by the above-described ranges, wherein these defined ranges are selected to provide reduced stickiness during and after application of the multi-phase personal care composition on hair or skin.

The Shear Index (n) and Consistency Value (K) are known and accepted means for reporting the viscosity profile of materials having a viscosity that varies with applied shear rate using a Power Law model.

The viscosity (μ) for a benefit phase can be measured by applying a shear stress and measuring the shear rate using a rheometer, such as a TA Instruments AR2000 (TA Instruments, New Castle, Del., USA 19720). Viscosity is determined at different shear rates in the following manner. First, the benefit phase is obtained. If there exists more than one distinct (immiscible, e.g.) benefit phase in the composition, such as for example a silicone oil phase and a hydrocarbon phase, they are prepared and evaluated separately from each other.

For measurement, a 40 mm diameter parallel plate geometry with a gap of 1 mm is used unless there are particles greater than 0.25 mm, in which case a gap of 2 mm is used. The rheometer uses standard parallel plate conventions to report shear rate at the edge as shear rate of the test; and converts torque to stress using the factor 2/(πR³). Using a spatula, a sample comprising a small excess of the benefit phase is loaded onto the rheometer base plate which is at 25° C., the gap is obtained, and excess composition outside the top measurement geometry is removed, locking the top plate in position during the removal of excess sample. The sample is equilibrated to the base plate temperature for 2 minutes. A preshear step is performed comprising 15 seconds of shear at a shear rate of 50 inverse seconds (1/sec). As is known to one skilled in the art, the shear rate with a parallel plate geometry is expressed as the shear rate at the edge, which is also the maximum shear rate. After the preshear step, the measurement is performed, which comprises ramping the stress from 10 Pa to 1,000 Pa over a 2.0 minute interval at 25° C., while collecting 60 viscosity data points, in an evenly spaced linear progression. A shear rate of at least 500 l/seconds is obtained in the test, or the test is repeated with a fresh sample of the same component with a higher final stress value, maintaining the same rate of stress increase per time, until a shear rate of at least 500 l/sec is obtained during the measurement period. During the measurement, observe the sample to make certain the area under the top parallel plate is not evacuated of sample at any edge location during the measurement, or the measurement is repeated until a sample remains for the duration of the test. If after several trials a result cannot be obtained due to sample evacuation at the edge, the measurement is repeated leaving an excess reservoir of material at the edge (not scraping). If evacuation still cannot be avoided, a concentric cylinder geometry is used with a large excess of sample to avoid air pockets during loading. The results are fitted to the power law model by selecting only the data points between 25-500 l/sec shear rate, viscosity in Pa-s, shear rate in 1/sec, and using a least squares regression of the logarithm of viscosity vs. the logarithm of shear rate to obtain values of K and n according to the Power Law equation:
μ=K(γ′)⁽ⁿ⁻¹⁾

The value obtained for the log-log slope is (n−1) where n is the Shear Index and the value obtained for K is the Consistency Value, expressed in units of in Pa-s.

The hydrophobic composition comprises hydrophobic materials. Nonlimiting examples of hydrophobic material suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, and combinations thereof.

Non-limiting examples of silicone oils suitable for use as hydrophobic materials herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use as hydrophobic materials herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as hydrophobic materials herein include acetylated monoglycerides.

Non-limiting examples of alkyl esters suitable for use as hydrophobic materials herein include isopropyl esters of fatty acids and long chain esters of long chain (i.e. C₁₀-C₂₄) fatty acids, e.g. cetyl ricinoleate, non-limiting examples of which incloude isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic materials herein include oleyl myristate, oleyl stearate, oleyl oleate, and combinations thereof.

Non-limiting examples of polyglycerin fatty acid esters suitable for use as hydrophobic materials herein include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic materials herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable hydrophobic materials include milk triglycerides (e.g., hydroxylated milk glyceride) and polyol fatty acid polyesters.

Still other suitable hydrophobic materials include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The benefit phase of the composition preferably can comprise one or more hydrophobic materials, wherein at least 20% by weight of the hydrophobic materials are selected from petrolatum, mineral oil, sunflower seed oil, micro-crystalline waxes, paraffins, ozokerite, polyethylene, polybutene, polydecene and perhydrosqualene dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, castor oil, soy bean oil, maleated soy bean oil, safflower oil, cotton seed oil, corn oil, walnut oil, peanut oil, olive oil, cod liver oil, almond oil, avocado oil, palm oil and sesame oil, and combinations thereof. More preferably, at least about 50% by weight of the hydrophobic materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic skin conditioning agent is preferably selected from: isopropyl palmitate, cetyl riconoleate, octyl isononanoate, octyl palmitate, isocetyl stearate, hydroxylated milk glyceride and combinations thereof.

Non-Lathering Structured Aqueous Phase

The stable multi-phase personal care compositions of the present invention can comprise a non-lathering structured aqueous phase. The non-lathering structured aqueous phase of the composition comprises a water structurant and water. The non-lathering structured aqueous phase can be hydrophilic and in a preferred embodiment the non-lathering structured aqueous phase is a hydrophilic gelled water phase. In addition, the non-lathering structured aqueous phase typically comprises less than about 5%, preferably less than about 3%, and more preferably less than about 1%, by weight of the non-lathering structured aqueous phase, of a surfactant. In one embodiment of the present invention, the non-lathering structured aqueous phase is free of lathering surfactant in the formulation.

The non-lathering structured aqueous phase of the present invention comprises from about 30% to about 99%, by weight of the non-lathering structured aqueous phase, of water. The non-lathering structured aqueous phase generally comprises more than about 50%, preferably more than about 60%, even more preferably more than about 70%, still more preferably more than about 80%, by weight of the non-lathering structured aqueous phase, of water.

The non-lathering structured aqueous phase will typically have a pH of from about 5 to about 9.5, more preferably about 7. The non-lathering structured aqueous phase can optionally comprise a pH regulator to facilitate the proper pH range.

A water structurant for the non-lathering structured aqueous phase can have a net cationic charge, net anionic charge, or neutral charge. In a preferred embodiment, the water structurant for the non-lathering structured aqueous phase has a net anionic charge.

The non-lathering structured aqueous phase of the present compositions can further comprise optional ingredients such as those described hereinafter. Preferred optional ingredients for the non-lathering structured aqueous phase include pigments, pH regulators, and preservatives. In one embodiment, the non-lathering structured aqueous phase comprises a water structurant (e.g. acrylates/vinyl isodecanoate crosspolymer), water, a pH regulator (e.g. triethanolamine), and a preservative (e.g. 1,3-dimethylol-5,5-dimethylhydantoin (“DMDMH” available from Lonza under the trade name GLYDANT®)).

A) Water Structurant

The non-lathering structured aqueous phase comprises from about 0.1% to about 30%, preferably from about 0.5% to about 20%, more preferably from about 0.5% to about 10%, and even more preferably from about 0.5% to about 5%, by weight of the non-lathering structured aqueous phase, of a water structurant.

The water structurant is typically selected from the group consisting of inorganic water structurants, charged polymeric water structurants, water soluble polymeric structurants, associative water structurants, and mixtures thereof.

Non-limiting examples of inorganic water structurants for use in the multi-phased personal care composition include silicas, clays such as synthetic silicates (Laponite XLG and Laponite XLS from Southern Clay), polymeric gellants such as polyacrylates, polyacrylamides, starches, modified starches, crosslinked polymeric gellants, copolymers, or mixtures thereof.

Non-limiting examples of charged polymeric water structurants for use in the multi-phased personal care composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), or mixtures thereof.

Non-limiting examples of water soluble polymeric structurants for use in the multi-phased personal care composition include cellulosic gel, hydroxypropyl starch phosphate (Structured XL from National Starch), polyvinyl alcohol, or mixtures thereof.

Non-limiting examples of associative water structurants for use in the multi-phased personal care composition include xanthum gum, gellum gum, pectin, alginate, or mixtures thereof.

Organic Cationic Deposition Polymer

The stable multi-phased personal care compositions of the present invention can additionally comprise an organic cationic deposition polymer in the cleansing phase as a deposition aid for the benefit agents described hereinafter. Concentrations of the cationic deposition polymer preferably range from about 0.025% to about 3%, more preferably from about 0.05% to about 2%, even more preferably from about 0.1% to about 1%, by weight of the cleansing phase composition.

Suitable cationic deposition polymers for use in the stable multi-phased personal care compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. The average molecular weight of the cationic deposition polymer is between about 5,000 to about 10 million, preferably at least about 100,000, more preferably at least about 200,000, but preferably not more than about 2 million, more preferably not more than about 1.5 million. The polymers also have a cationic charge density ranging from about 0.2 meq/gm to about 5 meq/gm, preferably at least about 0.4 meq/gm, more preferably at least about 0.6 meq/gm., at the pH of intended use of the personal cleansing composition, which pH will generally range from about pH 4 to about pH 9, preferably between about pH 5 and about pH 8.

The charge density can be controlled and adjusted in accordance with techniques well known in the art. As used herein the “charge density” of the cationic polymers is defined as the number of cationic sites per polymer gram atomic weight (molecular weight), and can be expressed in terms of meq/gram of cationic charge. In general, adjustment of the proportions of amine or quaternary ammonium moieties in the polymer, as well as pH of the multi-phased personal care compositions in the case of the amines, will affect the charge density.

Any anionic counterions can be use in association with the cationic deposition polymers so long as the polymers remain soluble in water, in the stable multi-phased personal care compositions, or in a coacervate phase of the stable multi-phased personal care compositions, and so long as the counterions are physically and chemically compatible with the essential components of the personal cleansing composition or do not otherwise unduly impair product performance, stability or aesthetics. Nonlimiting examples of such counterions include halides (e.g., chlorine, fluorine, bromine, iodine), sulfate and methlylsulfate.

Nonlimiting examples of cationic deposition polymers for use in the stable multi-phase personal care compositions include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M.

Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

The cationic polymers herein are either soluble in the cleansing phase, or preferably are soluble in a complex coacervate phase in the stable multi-phased personal care compositions formed by the cationic deposition polymer and the anionic surfactant component described hereinbefore. Complex coacervates of the cationic deposition polymer can also be formed with other charged charged materials, ionic and nonionic surfactants and/or ionic and nonionic polymers in the stable multi-phased personal care compositions.

Coacervate formation is dependent upon a variety of criteria such as molecular weight, component concentration, and ratio of interacting ionic components, ionic strength (including, modification of ionic strength, for example, by addition of salts), charge density of the cationic and anionic components, pH, and temperature. Coacervate systems and the effect of these parameters have been described, for example, by J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid anti Interface Science, Vol. 140, No. 1, November 1990, pp 227-238, which descriptions are incorporated herein by reference.

It is believed to be particularly advantageous for the cationic deposition polymer if present in the stable multi-phased personal care compositions in a coacervate phase, or to form a coacervate phase upon application or rinsing of the stable multi-phased personal care compositions to or from the skin. Complex coacervates are believed to more readily deposit on the skin, which results in improved deposition of the benefit materials. Thus, in general, it is preferred that the cationic deposition polymer exists in the stable multi-phased personal care compositions as a coacervate phase or form a coacervate phase upon dilution. If not already a coacervate in the stable multi-phased personal care compositions, the cationic deposition polymer will preferably exist in a complex coacervate form in the stable multi-phased personal care compositions upon dilution with water.

Techniques for analysis of formation of complex coacervates are known in the art. For example, centrifugation analyses of the multi-phased personal care compositions, at any chosen stage of dilution, can be utilized to identify whether a coacervate phase has formed.

Particle

The stable multi-phase personal care composition of the present invention can comprise a particle. Water insoluble solid particle of various shapes and densities is useful. In a preferred embodiment, the particle tends to have a spherical, an oval, an irregular, or any other shape in which the ratio of the largest dimension to the smallest dimension (defined as the Aspect Ratio) is less than about 10. More preferably, the Aspect Ratio of the particle is less than about 8, still more preferably the Aspect Ratio of the particle is less than about 5.

The particle of the present invention has a particle size (volume average based on the particle size measurement described hereafter) of less than about 10,000 um, preferably less than about 1,000 um, and more preferably less than 100 μm.

Some particle of the present invention preferably have a particle size of greater than about 0.1 μm, preferably a particle size of greater than about 0.5 μm, more preferably, a particle size greater than about 1 μm, still more preferably a particle size greater than about 2 μm, even more preferably a particle size greater than about 3 μm, and still even more preferably a particle size greater than about 4 μm.

The particle has a diameter from about 1 μm to about 70 μm, more preferably from about 2 μm to about 65 μm, and even more preferably from about 2 μm to about 60 μm in diameter.

The stable multi-phase personal care composition of the present invention comprises the particle at a cosmetically efficacious level. Preferably, the particles are present from at least about 0.1% by weight of the composition, more preferably at least about 0.2% by weight of composition, even more preferably at least about 0.5%, still more preferably at least about 1%, and even still more preferably at least 2% by weight of composition. In the multi-phase personal care composition of the present invention, preferably the particles comprises no more than about 50% by weight of composition, more preferably no more than about 30%, still more preferably no more than about 20%, and even more preferably no more than about 10% by weight of composition.

Preferably, the particle will also have physical properties which are not significantly affected by typical processing of the composition. Preferably, a particle having a melting point greater than about 70° C. is used, more preferably having a melting point greater than about 80° C., and even more preferably having a melting point of greater than about 95° C. is used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Non limiting examples of natural particles include various precipitated silica particles in hydrophilic and hydrophobic forms available from Degussa-Huls under the trade name Sipernet. Precipitated™, hydrophobic, synthetic amorphous silica, available from Degussa under the trade name Sipernet D11™ is a preferred particle. Snowtex colloidal silica particles available from Nissan Chemical America Corporation.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501 (nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of inorganic pigments include iron oxides, ferric ammonium ferrocyanide, manganese violet, ultramarine blue, Chrome oxide, and Chromoxide Green (from Sun Chemical).

Non limiting examples of hybrid particles include Ganzpearl GSC-30SR (Sericite & crosslinked polystyrene hybrid powder), and SM-1000, SM-200 (mica and silica hybrid powder available from Presperse).

Exfoliant Particle

The stable multi-phase personal care composition of the present invention can comprise an exfoliant particle that is selected from the group consisting of polyethylene, microcryatalline wax, jojoba esters, amourphors silica, talc, tracalcium orthophosphate, or blends thereof, and the like in at least one phase of the multi-phase personal care composition. The exfoliant particle has a particle size dimension along the major axis of the particle of from about 100 microns to about 600 microns, preferably from about 100 microns to about 300 microns. The exfoliant particle has a hardness of less than about 4 Mohs, preferably less than about 3 Mohs. The hardness as so measured is a criterion of the resistance of a particular material to crushing. It is known as being a fairly good indication of the abrasive character of a particulate ingredient. Examples of materials arranged in increasing order of hardness according to the Moh scale are as follows: h (hardness)-1:talc; h-2: gypsum, rock salt, crystalline salt in general, barytes, chalk, brimstone; h-4: fluorite, soft phosphate, magnesite, limestone; h-5: apatite, hard phosphate, hard limestone, chromite, bauxite; h-6: feldspar, ilmenite, hornblendes; h-7: quartz, granite; h-8: topaz; h-9: corrundum, emery; and h-10: diamond.

Preferably, the exfoliant particle has a color distinct from the cleansing base. The exfoliant particle is preferably present at a level of less than about 10%, preferably less than about 5%, by wt of the composition.

Shiny Particles

The stable multi-phase personal care compositions of the present invention can comprise a shiny particle in at least one phase of the multi-phase personal care composition. Nonlimiting examples of shiny particles include the following: interference pigment, multi-layered pigment, metallic particle, solid and liquid crystals, or combinations thereof.

An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer. The interference pigments of the multi-phased personal care compositions preferably comprises no more than about 20 weight percent of the composition, more preferably no more than about 10 weight percent, even more preferably no more than about 7 weight percent, and still more preferably no more than about 5 weight percent of the multi-phased personal care composition. The interference pigment of the multi-phased personal care composition preferably comprises at least about 0.1 weight percent of the multi-phased personal care composition, more preferably at least about 0.2 weight percent, even more preferably at least about 0.5 weight percent, and still more preferably at least about 1 weight percent by weight of the composition. When pigment is applied and rinsed as described in the Pigment Deposition Tape Strip Method as described in copending application Ser. No. 60/469,075 filed on May 8, 2003, the deposited pigment on the skin is preferably at least 0.5 μg/cm², more preferably at least 1 μg/cm², and even more preferably at least 5 μg/cm².

The interference pigments of the present invention are platelet particulates. The platelet particulates of the multi-phased personal care compositions preferably have a thickness of no more than about 5 μm, more preferably no more than about 2 μm, still more preferably no more than about 1 μm. The platelet particulates of the multi-phased personal care composition preferably have a thickness of at least about 0.02 μm, more preferably at least about 0.05 μm, even more preferably at least about 0.1 μm, and still more preferably at least about 0.2 μm.

The particle size determines the opacity and luster. The particle size is determined by measuring the diameter thickness of the particulate material. The term “diameter” as used herein, means the largest distance across the major axis of the particulate material. Diameter can be determined by any suitable method known in the art, such as particle size analyzer Mastersizer 2000 manufactured by Malvern Instruments. The interference pigment of the stable multi-phased personal care compositions preferably have an average diameter not greater than about 200 μm, more preferably not greater than 100 μm, even more preferably not greater than about 80 μm, still more preferably not greater than than about 60 μm. The interference pigment of the stable multi-phased personal care compositions preferably have a diameter of at least about 0.1 μm, more preferably at least about 1.0 μm, even more preferably at least about 2.0 μm, and still more preferably at least about 5.0 μm.

The interference pigment of the stable multi-phased personal care compositions can comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, zeolite, boron nitride, oxychloride, silica flake, glass flake, ceramics, titanium dioxide, CaSO₄, CaCO₃, BaSO₄, borosilicate and mixtures thereof, preferably mica, cellulose acetate, PTFE, modified starch, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃SnO₂. For the multiple layer structures, the thin films can be consisted of all high refractive index materials or alternation of thin films with high and low RI materials with the high RI film as the top layer.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine. It was found that the color of the interference pigment in addition to its brightness had a significant influence on human perception of skin tone. In general, preferred colors are silver, gold, red, green and mixtures thereof. In one preferred embodiment the human perception of skin tone is a whitening of ones skin tone.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The Particle Contact Angle Test as described in copending application Ser. No. 60/469,075 filed on May 8, 2003 is used to determine contact angle of interference pigments. The greater the contact angle, the greater the hydrophobicity of the interference pigment. The interference pigment of the present invention possess a contact angle of at least 60 degrees, more preferably greater than 80 degrees, even more preferably greater than 100 degrees, still more preferably greater than 100 degrees. The hydrophobically modified interference pigment or HMIP allows for the entrapment of the HMIP within the phases and greater deposition of the HMIP. Preferably the ratio of HMIP to a phase is 1:1 to about 1:70, more preferably 1:2 to about 1:50, still more preferably 1:3 to about 1:40 and most preferably 1:7 to about 1:35.

In an embodiment of the present invention the HMIP's are preferably entrapped within the benefit phase. This necessitates that the benefit phase particle size is generally larger than the HMIP. In a preferred embodiment of the invention, the benefit phase particles contain only a small number of HMIPs per benefit particles. Preferably this is less than 20, more preferably less than 10, most preferably less than 5. These parameters, the relative size of the benefit droplets to the HMIP and the approximate number of HMIP particles per benefit particles, can be determined by using visual inspection with light microscopy.

The HMIP and the benefit phase can be mixed into the composition via a premix or separately. For the case of separate addition, the hydrophobic pigments partition into the benefit phase during the processing of the formulation. The HMIP of the present invention preferably has a hydrophobic coating comprising no more than about 20 weight percent of the total particle weight, more preferably no more than about 15 weight percent, even more preferably no more than about 10 weight percent. The HMIP of the present invention preferably has a hydrophobic coating comprising at least about 0.1 weight percent of the total particle weight, more preferably at least about 0.5 weight percent, even more preferably at least about 1 weight percent. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

Skin Lightening Agents

The stable multi-phase personal care composition of the present invention can comprise a skin lightening agent. These skin lightening agent are preferably present at from about 0.0001% to about 20%, more preferably from about 0.05% to about 5%, even more preferably from about 0.1% to about 2%, by weight of the composition. Suitable skin lightening agents include those known in the art, including kojic acid, arbutin, tranexamic acid, ascorbic acid and derivatives thereof (e.g., magnesium ascorbyl phosphate or sodium ascorbyl phosphate, ascorbyl glucoside, and the like). Other skin lightening materials suitable for use herein include Actiwhite® (Cognis), Emblica® (Rona), Azeloglicina (Sinerga), Sepiwhite, hexamidine, sugar amines, (e.g., N-acetyl glucosamine), phytosterols (e.g. one or more sitosterol, stigmasterol, campesterol, brassicasterol, etc.), and extracts (e.g. mulberry extract).

Beads

The stable multi-phase personal care composition of the present invention can comprise beads. These beads are preferably present at from about 0.01% to about 10%, more preferably from about 0.1% to about 5%, even more preferably from about 0.5% to about 3%, by weight of the composition. The beads may be any color. The beads may be located in one phase or multiple phase of the of the stable multi-phase personal care composition. Beads may be used for signals for whitening, moisturizing, anti-aging, cleansing, exfoliation, scent bloom, scent longevity, and carriers for optional ingredients listed herein. Suitable beads include those known in the art, including soft and hard beads. Suitable examples of soft beads include unispheres, made by Induchem, Unispheres NT-2806 (Pink). Suitable examples of hard beads include polyethylene, oxidized polyethylene, preferably those made by Accutech.

Optional Ingredients

A variety of suitable optional ingredients can be employed in the stable multi-phase personal care composition. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional materials can be used in any aspect of the compositions of the present invention, including each phase as described herein.

Non-limiting optional ingredients include humectants and solutes. A variety of humectants and solutes can be employed and can be present at a level of from about 0.1% to about 50%, preferably from about 0.5% to about 35%, and more preferably from about 2% to about 20%, by weight of the personal care composition. A preferred humectant is glycerin.

A preferred water soluble, organic material is selected from the group consisting of a polyol of the structure:
R1-O(CH₂—CR2HO)ₙH
where R1=H, C1-C4 alkyl; R2=H, CH₃ and n=1-200; C2-C10 alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)ₓNH_{y} where x=1-3; y=0-2, and x+y=3, and mixtures thereof. The most preferred polyols are selected from the group consisting of glycerine, polyoxypropylene(1) glycerol and polyoxypropylene(3) glycerol, sorbitol, butylene glycol, propylene glycol, sucrose, urea and triethanol amine.

Nonionic polyethylene/polypropylene glycol polymers are preferably used as skin conditioning agents. Polymers useful herein that are especially preferred are PEG-2M wherein x equals 2 and n has an average value of about 2,000 (PEG 2-M is also known as Polyox WSR® N-10 from Union Carbide and as PEG-2,000); PEG-SM wherein x equals 2 and n has an average value of about 5,000 (PEG 5-M is also known as Polyox WSR® 35 and Polyox WSR® N-80, both from Union Carbide and as PEG-5,000 and Polyethylene Glycol 200,000); PEG-7M wherein x equals 2 and n has an average value of about 7,000 (PEG 7-M is also known as Polyox WSR® (N-750 from Union Carbide); PEG-9M wherein x equals 2 and n has an average value of about 9,000 (PEG 9-M is also known as Polyox WSR® N-3333 from Union Carbide); PEG-14 M wherein x equals 2 and n has an average value of about 14,000 (PEG 14-M is also known as Polyox WSR-205 and Polyox WSR® N-3000 both from Union Carbide); and PEG-90M wherein x equals 2 and n has an average value of about 90,000. (PEG-90M is also known as Polyox WSR®-301 from Union Carbide.)

Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Yield Stress and Zero Shear Viscosity Mehtods

The cleansing phase comprises a surfactant component that is measured either prior to combining in the composition, or after combining in the composition by separating the surfactant component by suitable physical separation means, such as centrifugation, pipetting, cutting away mechanically, rinsing, filtering, or other separation means.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress.

First a surfactant component is obtained and placed in position on the rheometer base plate, the measurement geometry (upper plate) moving into position 1 mm above the base plate. Excess surfactant component at the geometry edge is removed by scraping after locking the geometry. If the surfactant component comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a surfactant component has any particle larger than 5 mm in any dimension, the component between the particles is measured by removing the particles prior to measuring the component.

The determination is performed via the programmed application of a continuous shear stress ramp from 0.1 Pa to 1,000 Pa over a time interval of 5 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the average of the 3 highest viscosity values obtained in the test, the yield stress is zero, and the following mathematical procedure is not used. A straight line regression is performed on the results using the logarithmically transformed data in the indicated stress region, an equation being obtained of the form:
Log(strain)=m*Log(stress)+b (1)

Using the regression obtained, for each stress in the determination between 0.1 and 1,000 Pa, a predicted value of log(strain) is obtained using the coefficients m and b obtained, and the actual stress in Equation (1). From the predicted log(strain), a predicted strain at each stress is obtained by taking the antilog (i.e., 10^{x} for each x. The predicted strain is compared to the actual strain at each measurement point to obtain a % variation at each point, using Equation (2).
% variation=100*(measured strain−predicted strain)/measured strain (2)

The Yield Stress is the first stress (Pa) at which % variation exceeds 10%, and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

Lather Volume Test

Lather volume of a cleansing phase, a surfactant component or a structured domain of a stable, patterned multi-phased personal care composition, is measured using a graduated cylinder and a rotating apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 25° C.) is added to the graduated cylinder. The cylinder is clamped in a rotating device, which clamps the cylinder with an axis of rotation that transects the center of the graduated cylinder. Inject 0.50 grams of a surfactant component or cleansing phase from a syringe (weigh to ensure proper dosing) into the graduated cylinder onto the side of the cylinder, above the water line, and cap the cylinder. When the structured domain is evaluated, use only 0.25 cc, keeping everything else the same. The cylinder is rotated for 20 complete revolutions at a rate of about 10 revolutions per 18 seconds, and stopped in a vertical position to complete the first rotation sequence. A timer is set to allow 15 seconds for lather generated to drain. After 15 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells which comprise the lather (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum lather height is 1,000 ml (even if the total lather height exceeds the 1,000 ml mark on the graduated cylinder). 30 seconds after the first rotation is completed, a second rotation sequence is commenced which is identical in speed and duration to the first rotation sequence. The second lather volume is recorded in the same manner as the first, after the same 15 seconds of drainage time. A third sequence is completed and the third lather volume is measured in the same manner, with the same pause between each for drainage and taking the measurement.

The lather result after each sequence is added together and the Total Lather Volume determined as the sum of the three measurements, in ml. The Flash Lather Volume is the result after the first rotation sequence only, in ml, i.e., the first lather volume. Compositions according to the present invention perform significantly better in this test than similar compositions in conventional emulsion form.

Ultracentrifugation Method:

The Ultracentrifugation Method is used to determine the percent of a structured domain or an opaque structured domain that is present in a stable multi-phased personal care composition that comprises cleansing phase comprising a surfactant component. The method involves the separation of the composition through ultracentrifugation into separate but distinguishable layers. The stable multi-phased personal care composition of the present invention can have multiple distinguishable layers, for example a non-structured surfactant layer, a structured surfactant layer, and a benefit layer.

First, dispense about 4 grams of stable multi-phased personal care composition into Beckman Centrifuge Tube (11×60 mm). Next, place the centrifuge tubes in an Ultracentrifuge (Beckman Model L8-M or equivalent) and set ultracentrifuge to the following conditions: 50,000 rpm, 18 hours, and 25 C.

After ultracentrifuging for 18 hours, determine the relative phase volume by measuring the height of each layer using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:
Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated based on the following equation:
Structured Domain Volume Ratio=H_{c}/Hₛ*100%

If there is no benefit phase present, use the total height as the surfactant layer height, Hₛ=Hₐ.

Method Of Use

The stable multi-phase personal care compositions of the present invention are preferably applied topically to the desired area of the skin or hair in an amount sufficient to provide effective delivery of the skin cleansing agent, hydrophobic material, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water.

The present invention is therefore also directed to methods of cleansing the skin through the above-described application of the compositions of the present invention. The methods of the present invention are also directed to a method of providing effective delivery of the desired skin active agent, and the resulting benefits from such effective delivery as described herein, to the applied surface through the above-described application of the compositions of the present invention.

Method Of Manufacture

The stable multi-phase personal care compositions of the present invention may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples.

If the stable multi-phase personal care compositions contain patterns of varying colors it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

The following cleansing phases (Examples 1-21) are prepared as non-limiting examples.

| | Ex. 1 | | | | | | |
|---|---|---|---|---|---|---|---|
| Cleansing Phase | (reference) | Ex. 2 | Ex. 3 | Ex. 4 | Ex. 5 | Ex. 6 | Ex. 7 |
| Skin Benefit Components | | | | | | | |
| and Thickeners | | | | | | | |
| Water, distilled | QS | QS | QS | QS | QS | QS | QS |
| Glycerin | 0.80 | 0.30 | 0.41 | 0.30 | 0.30 | 0.17 | 0.60 |
| Guar hydroxypropropyltrimonium | 0.70 | 0.28 | 0.59 | 0.33 | 0.40 | 0.43 | 0.50 |
| chloride(N- | | | | | | | |
| Hance 3196, Aqualon | | | | | | | |
| Chem.) | | | | | | | |
| PEG 90M (Polyox WSR | 0.20 | | | | 0.10 | 0.05 | |
| 301, Amerchol Corp) | | | | | | | |
| Citric acid | 0.40 | | | | | 0.46 | |
| Surfactant Components | | | | | | | |
| Sodium trideceth sulfate | | | | | | 9.9 | |
| (Cedepal TD-403, Stepan | | | | | | | |
| Co.) | | | | | | | |
| Ammonium Lauryl | | 10.69 | 13.36 | 6.00 | 9.40 | | |
| Sulfate (The Procter & | | | | | | | |
| Gamble Co.) | | | | | | | |
| Sodium Lauryl Sulfate | | | | | | | 14.69 |
| (The Procter & Gamble | | | | | | | |
| Co.) | | | | | | | |
| Miracare SLB-365 | 23.70 | | | 8.00 | | | |
| (Rhodia, Inc.) (Sodium | | | | | | | |
| Trideceth Sulfate, Sodium | | | | | | | |
| Lauramphoacetate, | | | | | | | |
| Cocamide MEA) | | | | | | | |
| Polyoxyethylene 2.5 | | 2.37 | 2.96 | | 2.10 | 1.3 | 3.26 |
| lauryl alcohol (Arylpon F, | | | | | | | |
| Cognis Corp, Cincinnati, | | | | | | | |
| OH) | | | | | | | |
| Cocamide | | | | 2.00 | | | |
| monoethanolamine | | | | | | | |
| Cocamidopropyl betaine | | 2.96 | 3.68 | | 2.60 | 4.8 | 4.05 |
| (Tegobetaine F, DeGussa) | | | | | | | |
| Preservative and Minors | | | | | | | |
| Fragrance | 1.4 | 1.33 | 1.25 | 1.33 | 1.40 | 1.25 | 2.00 |
| Sodium chloride | 3.50 | 2.33 | 3.50 | 2.33 | 3.50 | 3.50 | 3.00 |
| Disodium EDTA | 0.05 | | | | | | |
| Preservative | 0.4 | 0.1 | 0.1 | 0.1 | 0.4 | 0.4 | 0.1 |
| Polymeric Phase | | | | | | | |
| Structurants | | | | | | | |
| Xanthan gum (Keltrol | | 0.33 | 0.33 | 0.33 | 0.26 | 0.50 | 0.35 |
| CGT from Kelco) | | | | | | | |
| Acrylates/Vinyl | | 0.67 | 0.53 | 0.67 | 0.54 | 0.50 | 0.35 |
| Isodecanoate | | | | | | | |
| Crosspolymer | | | | | | | |
| (Stabylen 30 from 3V) | | | | | | | |
| Final pH (adjust using | 6.2 | 6.5 | 6.5 | 6.4 | 6.25 | 6.2 | 6.5 |
| NaOH or citric acid) | | | | | | | |
| Total surfactant solids, % | 23.70 | 16.02 | 20.0 | 16.0 | 14.1 | 16.0 | 22.0 |
| of phase (calculated) | | | | | | | |
| Zero shear viscosity, Pa- | 6,530 | 7,070 | 5,630 | 2,960 | 7,550 | 8,390 | 5,200 |
| sec | | | | | | | |
| Yield stress, Pa | 13.8 | 17 | 18 | 16 | 23.6 | 4.1 | 25.6 |
| Dissolution Rate | 73% | 63% | | | | | |
| Coacervate | <1 mm | | 4 ml | | | | 6 ml |
| Lather Volume: | 590/ | 460/ | 500/ | | 470/ | | 510/ |
| Flash/Total (ml/ml) | 2080 | 1780 | 1860 | | 1760 | | 1930 |
| Structured phase volume, % | 88 | | 86 | 86 | | | |

| | | | Ex. | Ex. | Ex. | Ex. | Ex. |
|---|---|---|---|---|---|---|---|
| Cleansing Phase | Ex. 8 | Ex. 9 | 10 | 11 | 12 | 13 | 14 |
| Skin Benefit | | | | | | | |
| Components and | | | | | | | |
| Thickeners | | | | | | | |
| Water, distilled | QS | QS | QS | QS | QS | QS | QS |
| Glycerin | | | 0.5 | 0.5 | 0.5 | 0.17 | 0.17 |
| Guar hydroxypropropyltrimonium | 0.49 | 0.45 | 0.45 | 0.45 | 0.45 | 0.43 | 0.43 |
| chloride(N- | | | | | | | |
| Hance 3196, Aqualon | | | | | | | |
| Chem.) | | | | | | | |
| PEG 90M (Polyox WSR | | | 0.08 | 0.08 | 0.08 | 0.05 | 0.05 |
| 301, Amerchol Corp) | | | | | | | |
| Citric acid | | | 0.2 | 0.2 | 0.2 | 0.46 | 0.46 |
| Surfactant Components | | | | | | | |
| Sodium trideceth sulfate | | | | | | 5.26 | |
| (Cedepal TD-403, | | | | | | | |
| Stepan Co.) | | | | | | | |
| Ammonium Lauryl | | | | | | 6.1 | 8.0 |
| Sulfate (The Procter & | | | | | | | |
| Gamble Co.) | | | | | | | |
| Ammonium Laureth | 14.1 | | | | | | |
| Sulfate (The Procter & | | | | | | | |
| Gamble Co.) | | | | | | | |
| Sodium Laureth Sulfate | | 15.1 | | | | | |
| (2 mole ethoxy, P&G) | | | | | | | |
| Miracare SLB-365 | | | 15.72 | 15.72 | 15.72 | | 9.0 |
| (Rhodia, Inc.) (Sodium | | | | | | | |
| Trideceth Sulfate, | | | | | | | |
| Sodium Lauramphoacetate, | | | | | | | |
| Cocamide | | | | | | | |
| MEA) | | | | | | | |
| Polyoxyethylene 2.5 | 2.3 | 2.1 | | | | 2.1 | |
| lauryl alcohol (Arylpon | | | | | | | |
| F, Cognis Corp, | | | | | | | |
| Cincinnati, OH) | | | | | | | |
| Cocamidopropyl betaine | 2.83 | 2.62 | | | | 2.62 | 1.0 |
| (Tegobetaine F, | | | | | | | |
| DeGussa) | | | | | | | |
| Preservative and Minors | | | | | | | |
| Fragrance | 3.5 | 2.7 | 1.25 | 1.25 | 1.25 | 1.25 | 1.25 |
| Sodium chloride | 2.9 | 2.7 | 2.8 | 2.8 | 2.8 | 3.5 | 3.5 |
| Disodium EDTA | | | | | | | |
| Preservative | 0.1 | 0.1 | 0.25 | 0.25 | 0.25 | 0.3 | 0.3 |
| Polymeric Phase | | | | | | | |
| Structurants | | | | | | | |
| Xanthan gum (Keltrol | 0.83 | 0.59 | | | 0.5 | 0.5 | 0.5 |
| CGT from Kelco) | | | | | | | |
| Acrylates/Vinyl | 1.08 | 0.68 | 0.5 | 0.8 | 0.5 | 0.5 | 0.5 |
| Isodecanoate | | | | | | | |
| Crosspolymer | | | | | | | |
| (Stabylen 30 from 3V) | | | | | | | |
| Final pH (adjust using | 5.9 | 5.8 | 6.7 | 5.8 | 6.2 | 6.3 | 6.3 |
| NaOH or citric acid) | | | | | | | |
| Total surfactant solids, | 19.23 | 19.82 | 15.72 | 15.72 | 15.72 | 16.08 | 18.0 |
| % of phase (calculated) | | | | | | | |
| Zero Shear Viscosity, | 15,800 | 1,640 | 3,330 | 8,660 | 10,100 | 12,900 | 7,110 |
| Pa-sec | | | | | | | |
| Yield Stress, Pa | 9.5 | 5.5 | 3.7 | 46 | 25.5 | 14 | 12 |
| Dissolution Rate | | | 72% | 71% | 60% | | |
| Coacervate | | | | | 13 ml | | |
| Lather Volume: | 400/ | 450/ | 490/ | 460/ | | | |
| Flash/Total (ml/ml) | 1580 | 1750 | 1840 | 1800 | | | |

| Cleansing Phase | Ex. | Ex. | Ex. | Ex. | Ex. | Ex. | Ex. |
|---|---|---|---|---|---|---|---|
| | 15 | 16 | 17 | 18 | 19 | 20 | 21 |
| Skin Benefit | | | | | | | |
| Components and | | | | | | | |
| Thickeners | | | | | | | |
| Water, distilled | QS | QS | QS | QS | QS | QS | QS |
| Glycerin | 0.5 | 0.5 | 0.5 | 0.5 | 0.3 | 0.43 | 0.43 |
| Guar | 0.45 | 0.45 | 0.45 | 0.45 | 0.40 | 0.53 | 0.53 |
| hydroxypropropyltrimonium | | | | | | | |
| chloride(N-Hance | | | | | | | |
| 3196, Aqualon Chem.) | | | | | | | |
| PEG 90M (Polyox WSR | 0.08 | 0.08 | 0.08 | 0.08 | 0.10 | 0.15 | 0.15 |
| 301, Amerchol Corp) | | | | | | | |
| Citric acid | 0.2 | 0.2 | 0.2 | 0.2 | | 0.4 | 0.4 |
| Surfactant Components | | | | | | | |
| Miracare SLB-365 | 15.72 | 15.72 | 15.72 | 15.72 | | 17.8 | 17.8 |
| (Rhodia, Inc.) | | | | | | | |
| (Sodium Trideceth | | | | | | | |
| Sulfate, Sodium | | | | | | | |
| Lauramphoacetate, | | | | | | | |
| Cocamide MEA) | | | | | | | |
| Polyoxyethylene 2.5 | | | | | 3.0 | | |
| lauryl alcohol (Arylpon | | | | | | | |
| F, Cognis Corp, | | | | | | | |
| Cincinnati, OH) | | | | | | | |
| Cocamidopropyl betaine | | | | | 3.7 | | |
| (Tegobetaine F, | | | | | | | |
| DeGussa) | | | | | | | |
| Ammonium Lauryl | | | | | 13.4 | | |
| Sulfate (The Procter & | | | | | | | |
| Gamble Co.) | | | | | | | |
| Cocamide | | | | | | 2.25 | 2.25 |
| Monoethanolamine | | | | | | | |
| Preservative and Minors | | | | | | | |
| Fragrance | 1.25 | 1.25 | 1.25 | 1.25 | 1.4 | 1.5 | 2.25 |
| Sodium chloride | 2.8 | 2.8 | 2.8 | 2.8 | 3.5 | 3.4 | 3.4 |
| Disodium EDTA | | | | | 0.06 | 0.06 | 0.06 |
| Preservative | 0.25 | 0.25 | 0.25 | 0.25 | 0.39 | 0.4 | 0.4 |
| Triethanolamine | | | | | | 0.38 | 0.38 |
| Titanium dioxide | | | | | | 1.0 | 1.0 |
| Polymeric Phase | | | | | | | |
| Structurants | | | | | | | |
| Xanthan gum (Keltrol | 0.3 | 0.3 | 0.3 | 0.3 | 0.13 | 0.25 | 0.25 |
| CGT or Keltrol 1000 | | | | | | | |
| from Kelco) | | | | | | | |
| Carbomer (Carbopol | 0.5 | | | | | | |
| 980) | | | | | | | |
| Carbomer (Carbopol | | 0.5 | | | | | |
| 954) | | | | | | | |
| Carbomer (Carbopol | | | 0.5 | | | | |
| 940) | | | | | | | |
| Acrylates copolymer | | | | 0.5 | | | |
| (Carbopol Aqua SF-1) | | | | | | | |
| Acrylates/Vinyl | | | | | 0.27 | 0.25 | 0.25 |
| Isodecanoate | | | | | | | |
| Crosspolymer (Stabylen | | | | | | | |
| 30 from 3V) | | | | | | | |
| Final pH (adjust using | 6 | 6.1 | 6.1 | 6.4 | 6.25 | | |
| NaOH or citric acid) | | | | | | | |
| Total surfactant solids, | 15.72 | 15.72 | 15.72 | 15.72 | 20.1 | | |
| % of phase (calculated) | | | | | | | |
| Zero Shear Viscosity, | 5,830 | 3,930 | 6,670 | 2,810 | 8,640 | | |
| Pa-sec | | | | | | | |
| Yield Stress, Pa | 2.4 | 4.1 | 2.6 | 16.6 | 13.8 | | |
| Dissolution Rate | 75% | 77% | 69% | 56% | 64% | | |
| Lather Volume: | | | | | | | |
| Flash/Total | | 470/ | 400/ | | 510/ | | |
| (ml/ml) | | 1830 | 1590 | | 1850 | | |

The cleansing phase can be prepared by conventional formulation and mixing techniques. Prepare the cleansing phase by first adding the water and skin benefit components and thickeners into a mixing vessel and agitate until a homogeneous dispersion is formed. Then add in the following sequence: surfactants, Disodium EDTA, preservative and half the sodium chloride and all other preservatives and minors except fragrance and the withheld sodium chloride. Heat to 65-70° C. if Cocaminde monoethanolamine is used, otherwise maintain at ambient temperature while agitating the mixing vessel. Cool to 45 C if heating was used. For additional stability, gas filled microspheres having a density of about 30 kg/m³ such as Expancel 091 DE 40 d30 (from Expancel, Inc.) can optionally be used at about 0.1-0.5% of the batch. In a separate vessel, prewet the structuring polymers with fragrance and add to the mix vessel at the same time as the remaining sodium chloride while agitating. Keep agitation until homogeneous, then pump through a static mixing element to disperse any polymer lumps to complete the batch. Coacervate amount is measured by thoroughly mixing (shake) 23 ml distilled water with 2 ml surfactant in a 25 ml graduated cylinder (e.g., Pyrex No. 3255) and allowing it to stand undisturbed for 1 week at 75° F., then observing the amount of turbid phase at the bottom, measuring in ml or if less than 1 ml, measuring in height from the bottom.

### Examples 22-30

For the following examples 22-30, the cleansing phase which is Example 1 is prepared except fragrance is withheld from the composition. The composition is denoted Fragrance Free Cleansing Phase 1 in the following examples and is shown as total weight added, not chemical weight. Examples 22-27 are prepared by prewetting the polymer component with the fragrance, blending the polymer-fragrance mixture with an equal weight of the fragrance free cleansing phase by hand using a spatula to prepare a paste, adding the remaining cleansing phase and stirring, adding additional water last and stirring by hand in small quantities (e.g., 75 gm total being prepared in about a 5 minute period). After preparation, the Examples are examined and found to be free of detectible lumps by eye and to the touch. Examples 28-30 are prepared by dispersing the polymer in water with high shear until free of lumps, then blending the mixture by vigorous hand stirring with the fragrance free cleansing phase and fragrance until homogeneous, about 2 minutes. The example compositions are then lightly centrifuged (3 min, 2,500 rpm in the mix jars) to deareate.

| | Ex. | | | | | | | | |
|---|---|---|---|---|---|---|---|---|---|
| | 22 | Ex. | Ex. | Ex. | Ex. | Ex. | Ex. | Ex. | Ex. |
| | (control) | 23 | 24 | 25 | 26 | 27 | 28 | 29 | 30 |
| Fragrance | 73.75 | 70.8 | 70.8 | 70.8 | 70.8 | 70.8 | 65.47 | 65.57 | 65.57 |
| Free | | | | | | | | | |
| Cleansing | | | | | | | | | |
| Phase 1 | | | | | | | | | |
| Fragrance | 0.625 | 0.60 | 0.60 | 0.60 | 0.60 | 0.60 | 1.64 | 1.64 | 1.64 |
| Hydroxy | | 4.0 | | | | | | | |
| propyl | | | | | | | | | |
| starch | | | | | | | | | |
| phosphate | | | | | | | | | |
| (Structure | | | | | | | | | |
| XL, | | | | | | | | | |
| National | | | | | | | | | |
| Starch | | | | | | | | | |
| Co.) | | | | | | | | | |
| Guar gum | | | 4.0 | | | | | | |
| (Supercol | | | | | | | | | |
| U, | | | | | | | | | |
| Hercules | | | | | | | | | |
| Inc. | | | | | | | | | |
| Aqualon | | | | | | | | | |
| Div.) | | | | | | | | | |
| Hydroxy- | | | | 4.0 | | | | | |
| ethyl- | | | | | | | | | |
| cellulose | | | | | | | | | |
| (250MR, | | | | | | | | | |
| Aqualon) | | | | | | | | | |
| Carboxy | | | | | 4.0 | | | | |
| methyl- | | | | | | | | | |
| cellulose | | | | | | | | | |
| (9M31XF, | | | | | | | | | |
| Aqualon) | | | | | | | | | |
| Starch | | | | | | 4.0 | | | |
| octenyl- | | | | | | | | | |
| succinate | | | | | | | | | |
| (NCreamer46, | | | | | | | | | |
| National | | | | | | | | | |
| Starch) | | | | | | | | | |
| PEG-150/ | | | | | | | 0.98 | | |
| Decyl | | | | | | | | | |
| Alcohol/ | | | | | | | | | |
| SMDI | | | | | | | | | |
| Copolymer | | | | | | | | | |
| (Aculyn | | | | | | | | | |
| 44, Rohm | | | | | | | | | |
| & Haas) | | | | | | | | | |
| Cetyl | | | | | | | | 0.66 | |
| hydroxyl- | | | | | | | | | |
| ethyl- | | | | | | | | | |
| cellulose | | | | | | | | | |
| (CS330, | | | | | | | | | |
| Aqualon) | | | | | | | | | |
| PEG- | | | | | | | | | 0.16 |
| 180/Laureth- | | | | | | | | | |
| 50/TMMG | | | | | | | | | |
| Copolymer | | | | | | | | | |
| (Pure | | | | | | | | | |
| Thix | | | | | | | | | |
| 1450, | | | | | | | | | |
| Sud- | | | | | | | | | |
| Chemie) | | | | | | | | | |
| Distilled | 25.625 | 24.6 | 24.7 | 24.7 | 24.7 | 24.7 | 31.91 | 32.13 | 32.63 |
| Water | | | | | | | | | |
| % Surfactant | 17.73 | 17.02 | 17.02 | 17.02 | 17.02 | 17.02 | 15.74 | 15.76 | 15.76 |
| component | | | | | | | | | |
| (calculated) | | | | | | | | | |
| Zero | 4,480 | 11,020 | 9,950 | 12,300 | 11,800 | 5,380 | 13,580 | 6,810 | 6,800 |
| Shear | | | | | | | | | |
| Viscosity | | | | | | | | | |
| (Pa-sec) | | | | | | | | | |
| Yield | 1.8 | 6.0 | 20.3 | 21.0 | 20.0 | 1.8 | 17.0 | 13.0 | 23.0 |
| Stress | | | | | | | | | |
| (Pa) | | | | | | | | | |

Non-Lathering Structured Aqueous Phase

The Non-Lathering Structured Aqueous Phase of Examples 31-32 can be prepared by dispersing polymers in water with high shear, adding salt and remaining ingredients except petrolatum and mineral oil, neutralizing to pH 7.0 with triethanolamine (approximate TEA level is shown), heating to 50° C., adding the petrolatum and mineral oil as a liquid at 80° C., and agitating until homogeneous without high shear. Pigments having no water soluble components are preferably used. A particle size of about 5-100 microns for the petrolatum component is obtained for most of the particles.

| Non-Lathering Structured | Example | Example |
|---|---|---|
| Aqueous Phase | 31 | 32 |
| Water, distilled | QS | QS |
| Acrylates/Vinyl Isodecanoate | 1.0 | 0.8 |
| Crosspolymer (Stabylen 30 | | |
| from 3V) | | |
| Xanthan gum (Keltrol CGT or | 1.0 | 0.8 |
| Keltrol 1000 from Kelco) | | |
| DMDM Hydantoin, | 0.4 | 0.4 |
| preservative | | |
| EDTA | 0.05 | 0.04 |
| Mineral oil (Hydrobrite 1000, | 0.03 | 4.82 |
| Witco) | | |
| Petrolatum (Super White | 20.0 | 18.78 |
| Protopet, Witco) | | |
| Petrolatum (G2218, Witco) | | |
| Triethanolamine | 0.80 | 0.80 |
| Sodium chloride | 3.0 | 2.4 |
| Pigment | 0.35 | 0.35 |

Benefit Phase

Benefit phases can be prepared having the following ingredients. The benefit phase of Examples 33-35 can be prepared by adding petrolatum into a mixing vessel. Heat to 190° F. (88° C.). Then, add mineral oil and particles. High shear the batch to ensure good pigment dispersion. Keep agitating the batch and slowly cool down the batch to ambient temperature. Pigments having no water soluble components are preferably used. A particle size of about 5-100 microns for the petrolatum component is obtained for most of the particles.

| Benefit Phase | Example 33 | Example 34 | Example 35 |
|---|---|---|---|
| Water, distilled | | | |
| Acrylates/Vinyl Isodecanoate | | | |
| Crosspolymer (Stabylen 30 | | | |
| from 3V) | | | |
| Xanthan gum (Keltrol CGT or | | | |
| Keltrol 1000 from Kelco) | | | |
| DMDM Hydantoin, | | | |
| preservative | | | |
| EDTA | | | |
| Mineral oil (Hydrobrite 1000, | | 30.0 | 30.0 |
| Witco) | | | |
| Petrolatum (Super White | | | 69.95 |
| Protopet, Witco) | | | |
| Petrolatum (G2218, Witco) | 99.95 | 69.95 | |
| Triethanolamine | | | |
| Sodium chloride | | | |
| Pigment | 0.05 | 0.05 | 0.05 |

Petrolatum can be obtained from Witco division of Crompton Corporation (Petrolia, Pa., USA). G2218 petrolatum has a complete melting point of about 139 degrees Fahrenheit, a Saybold viscosity of between about 75-86 SUS at 210° F., a Penetration of between 192-205 dmm, a Consistency Value of about 42 Pa-s with a shear index of about 0.53, a Structure Rigidity of 370 Pa and a Flow Onset Temperature of 109.8° F. A gas chromatogram of the petrolatum indicates hydrocarbons between C20 and C120 are present. Taking the ratio of the average peak heights of the GC for hydrocarbons having even numbered chain lengths from C22-28, C44-50 and C94-116, the petrolatum has a ratio of peak heights of about 0.72:1.0:0.32. Hydrobrite 1000 has a high viscosity relative to nearly all mineral oils.

Compositions

Patterned and stable multi-phase personal care compositions can be prepared by the following procedure. The benefit phase is lipid continuous, the benefit phase is maintained at 80° C. in a separate tank which is recirculated through a scraped wall heat exchanger having an outlet temperature of 45° C. Lipid at 45° C. is pumped either to the filling operation or back to the recirculation tank. The Non-Lathering Structured Aqueous Phase is water continuous, it is maintained in a hopper and gravity fed to the filling operation. Cleansing phase is maintained at ambient temperature in a gravity fed tank above the filler. Cleansing Phase and Benefit Phase or Non-Lathering Structured Aqueous Phase are simultaneously pumped in specified volumetric ratio through ¾ in. diameter pipes containing a 4-element static mixer (Koch/SMX type), the single pipe exits into a 10 oz. bottle on a spinning platform. The platform is set to 325 rpm spin speed, the composition filling 315 ml in about 2.0 seconds, the spinning platform being lowered during filling so that filling proceeds in a layering fashion from bottom to top. An even, relatively horizontal striped pattern is obtained. By adjusting temperature and viscosity of the phases, static mixer element types and number of elements, pipe diameters, spin rates, etc., a wide variety of patterns can be obtained.

Additionally, the present invention can be prepared by the method and apparatus as disclosed in U.S. Pat. No. 6,213,166 which method and apparatus allows two or more compositions to be filled with a spiral configuration into a single container using at least 2 nozzles.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable multi-phase personal care composition comprising: at least two visually distinct phases;
wherein at least one visually distinct phase comprises a cleansing phase comprising from about 2% to about 90%, by weight of the cleansing phase, of a surfactant component; and
wherein said cleansing phase comprises a polymeric phase structurant; and
wherein said visually distinct phases form a pattern.

2. The stable multi-phase personal care composition of claim 1, wherein said polymeric phase structurant is selected from the group consisting of deflocculating polymers, naturally derived polymers, synthetic polymers, crosslinked polymers, block polymers, block copolymers, copolymers, hydrophilic polymers, nonionic polymers, anionic polymers, hydrophobic polymers, hydrophobically modified polymers, associative polymers, oligomers, and mixtures thereof.

3. The stable multi-phase personal care composition of claim 1, wherein said multi-phase personal care composition comprises from about 0.05% to about 10%, by weight of said cleansing phase, of said polymeric phase structurant.

4. The stable multi-phase personal care composition of claim 1, wherein said cleansing phase further comprises a liquid crystalline phase inducing structurant.

5. The stable multi-phase personal care composition of claim 4, wherein said liquid crystalline phase inducing structurant is selected from the group consisting of fatty acids, fatty alcohols, fatty esters, trihydroxystrearin, and mixtures thereof.

6. The stable multi-phase personal care composition of claim 1, said cleansing phase comprising from about 5% to about 40%, by weight of the cleansing phase, of said surfactant component.

7. The stable multi-phase personal care composition of claim 1, cleansing phase comprising from about 10% to about 30%, by weight of the cleansing phase, of said surfactant component.

8. The stable multi-phase personal care composition of claim 1, cleansing phase comprising from about 12% to about 25%, by weight of the cleansing phase, of said surfactant component.

9. The stable multi-phase personal care composition of claim 1, cleansing phase comprising from about 13% to about 20.5%, by weight of the cleansing phase, of said surfactant component.

10. The stable multi-phase personal care composition of claim 1, cleansing phase comprising from about 14% to about 18.5%, by weight of the cleansing phase, of said surfactant component.

11. The stable multi-phased personal care composition of claim 1, wherein said visually distinct phases are selected from the group consisting of a cleansing phase, a benefit phase, a non-lathering structured aqueous phase, and combinations thereof.

12. The stable multi-phased personal care composition of claim 1, wherein a ratio of said cleansing phase to a second phase is about 99:1 to about 1:99.

13. The stable multi-phased personal care composition of claim 1, wherein a ratio of said cleansing phase to a second phase is about 90:10 to about 10:90.

14. The stable multi-phased personal care composition of claim 1, wherein a ratio of said cleansing phase to a second phase is about 80:20 to about 20:80.

15. The stable multi-phased personal care composition of claim 1, wherein a ratio of said cleansing phase to a second phase is about 70:30 to about 30:70.

16. The stable multi-phased personal care composition of claim 1, wherein a ratio of said cleansing phase to a second phase is about 50:50.

17. The stable multi-phase personal care composition of claim 1, wherein the cleansing phase comprises:
(i) at least one anionic surfactant;
(ii) at least one electrolyte;
(iii) at least one alkanolamide; and
(v) water;
wherein the cleansing phase is non-Newtonian shear thinning; and
the cleansing phase has a viscosity of equal to or greater than about 3000 cps.

18. The stable multi-phase personal care composition of claim 17, further comprising optionally additional conventional surfactants.

19. The stable multi-phase personal care composition of claim 1, wherein the cleansing phase comprises:
a. a surfactant component comprising:
(i) at least one nonionic surfactant having an HLB from about 3.4 to about 15.0;
(ii) at least one anionic surfactant;
(iii) at least one amphoteric surfactant; and
b. an electrolyte.

20. The stable multi-phase personal care composition of claim 1, wherein said pattern is selected from the group consisting of striped, geometric, marbled and combinations thereof.

21. The stable multi-phase personal care composition of claim 1, wherein said cleansing phases provides a Yield Stress of greater than about 0.5 Pa-s.

22. The stable multi-phase personal care composition of claim 1, wherein said cleansing phases provides a Zero Shear Viscosity of greater than about 500 Pa-s.

23. The stable multi-phased personal care composition of claim 1, wherein said cleansing phase provides a Total Lather Volume of at least about 600 ml.

24. The stable multi-phase personal cleansing composition of claim 1, wherein said composition additionally comprises optional benefit component, wherein said optional benefit component are selected from the group consisting of emollients, particles, beads, skin whitening agents, fragrances, colorants, of vitamins and derivatives thereof; sunscreens; preservatives; anti-acne medicaments; antioxidants; chelators; essential oils, skin sensates, antimicrobials, and mixtures thereof.

25. A stable multi-phase personal care composition comprising: at least two visually distinct phases;
wherein at least one visually distinct phase comprises a cleansing phase comprising from about 2% to about 90%, by weight of the cleansing phase, of a surfactant component; and
wherein said cleansing phase has a Structured Domain Volume Ratio of at least about 45%.

26. The stable multi-phase personal care composition of claim 25, wherein said composition further comprises a polymeric phase structurant.

27. The stable multi-phase personal care composition of claim 26, wherein said polymeric phase structurant is selected from the group consisting of deflocculating polymers, naturally derived polymers, synthetic polymers, crosslinked polymers, block polymers, block copolymers, copolymers, hydrophilic polymers, nonionic polymers, anionic polymers, hydrophobic polymers, hydrophobically modified polymers, associative polymers, oligomers, and mixtures thereof.

28. The stable multi-phase personal care composition of claim 26, wherein said cleansing phase comprises said polymeric phase structurant.

29. The stable multi-phase personal care composition of claim 26, wherein said multi-phase personal care composition comprises from about 0.05% to about 10%, by weight of said cleansing phase, of said phase structurant.

30. The stable multi-phase personal care composition of claim 25, wherein said cleansing phase further comprises a liquid crystalline phase inducing structurant.

31. The stable multi-phase personal care composition of claim 30, wherein said liquid crystalline phase inducing structurant is selected from the group consisting of fatty acids, fatty alcohols, fatty esters, trihydroxystrearin, and mixtures thereof.

32. The stable multi-phased personal care composition of claim 25, wherein said visually distinct phases are selected from the group consisting of a cleansing phase, a benefit phase, a non-lathering structured aqueous phase, and combinations thereof.

33. The stable multi-phase personal care composition of claim 25, wherein the cleansing phase comprises:
(i) at least one anionic surfactant;
(ii) at least one electrolyte;
(iii) at least one alkanolamide; and
(v) water;
wherein the cleansing phase is non-Newtonian shear thinning; and
the cleansing phase has a viscosity of equal to or greater than about 3000 cps.

34. The stable multi-phase personal care composition of claim 25, wherein said visually distinct phases form a pattern.

35. The stable multi-phase personal care composition of claim 34, wherein said pattern is selected from the group consisting of striped, geometric, marbled and combinations thereof.

36. The stable multi-phase personal cleansing composition of claim 25, wherein said composition additionally comprises optional benefit component, wherein said optional benefit component are selected from the group consisting of emollients, particles, beads, skin whitening agents, fragrances, colorants, of vitamins and derivatives thereof; sunscreens; preservatives; anti-acne medicaments; antioxidants; chelators; essential oils, skin sensates, antimicrobials, and mixtures thereof.

37. A multi-phase personal care composition comprising: at least two visually distinct phases comprising;
a) a first phase comprising a cleansing phase comprising from about 2% to about 90%, by weight of said cleansing phase, of a surfactant selected from the group consisting of anionic surfactant, nonionic surfactant, zwitterionic surfactant, cationic surfactant, soap, and mixtures thereof;
wherein said cleansing phase is non-Newtonian shear thinning, has a viscosity of equal to or greater than about 3,000 cps;
b) a benefit phase comprising a hydrophobic composition comprising from about 1% to about 100%, by weight of said benefit phase of a hydrophobic material is selected from the group consisting of lipids, hydrocarbons, fats, oils, hydrophobic plant extracts, fatty acids, essential oils, silicone oils, and mixtures thereof;
wherein a weight ratio between said cleansing phase and said benefit phase is from about 1:99 to about 99:1 and said cleansing phase and benefit phase are in physical contact in the same package and remain stable in ambient conditions for at least about 180 days; and wherein said cleansing phase and benefit phase form a pattern; and wherein at least one phase comprises a polymeric phase structurant; and wherein said visually distinct phase are stable.

38. The multi-phased personal care composition of claim 37, wherein said benefit phase is substantially free of surfactant.

39. The multi-phased phase personal cleansing composition of claim 37, wherein said hydrophobic material is selected from the group consisting of petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and combinations thereof.

40. The multi-phase personal cleansing composition of claim 37, wherein said composition additionally comprises optional benefit component, wherein said optional benefit component are selected from the group consisting of emollients, particles, beads, skin whitening agents, fragrances, colorants, vitamins and derivatives thereof; sunscreens; preservatives; anti-acne medicaments; antioxidants; chelators; essential oils, skin sensates, antimicrobials, and mixtures thereof.

41. The multi-phase personal care composition of claim 37, wherein said composition is packaged in a container such that said pattern is visible.

42. A multi-phase personal care composition comprising: at least two visually distinct phases comprising;
a) a first phase comprising a cleansing phase comprising from about 2% to about 90%, by weight of the cleansing phase, of a surfactant selected from the group consisting of anionic surfactant, non-ionic surfactant, zwitterionic surfactant, cationic surfactant, soap and mixtures thereof;
wherein said cleansing phase is non-Newtonian shear thinning, has a viscosity of equal to or greater than about 3,000 cps; and
b) a separate non-lathering structured aqueous phase; and
wherein the ratio of the cleansing phase to the non-lathering structured aqueous phase is from about 90:1 to about 1:90; wherein the cleansing phase and non-lathering structured aqueous phase are present as a pattern; and wherein at least one phase comprises a polymeric phase structurant; and wherein said visually distinct phase are stable.

43. The multi-phase personal cleansing composition of claim 42, wherein said composition additionally comprises optional benefit component, wherein said optional benefit component are selected from the group consisting of emollients, particles, beads, skin whitening agents, fragrances, colorants, of vitamins and derivatives thereof; sunscreens; preservatives; anti-acne medicaments; antioxidants; chelators; essential oils, skin sensates, and mixtures thereof.

44. The multi-phase personal care composition of claim 42, wherein said composition is packaged in a container such that said pattern is visible.

45. A method of delivering skin benefits to skin or hair, said method comprising the steps of:
a) dispensing an effective amount of a stable multi-phase personal care composition according to claim 1 onto an implement selected from the group consisting of a cleansing puff, washcloth, sponge, and human hand;
b) topically applying said composition to said skin or hair using said implement; and
c) removing said composition from said skin or hair by rinsing said skin or hair with water.

46. A method of delivering skin benefits to skin or hair, said method comprising the steps of:
a) dispensing an effective amount of a stable multi-phase personal care composition according to claim 25 onto an implement selected from the group consisting of a cleansing puff, washcloth, sponge, and human hand;
b) topically applying said composition to said skin or hair using said implement; and
c) removing said composition from said skin or hair by rinsing said skin or hair with water.
